(19) United States
(12) Patent Application Publication
(10) Pub. No.: US 2017/0348199 A1
(43) Pub. Date: Dec. 7, 2017

(54) ANESTHETIC AND ROOT CANAL PREPARATION LUBRICANT COMPOSITION

(71) Applicant: PINKBAND DENTAL SOLUTIONS, INC., Grayslake, IL (US)

(72) Inventor: Robert E. Haraden, Libertyville, IL (US)

(21) Appl. No.: 15/615,601

(22) Filed: Jun. 6, 2017

Related U.S. Application Data
(60) Provisional application No. 62/345,952, filed on Jun. 6, 2016.

(51) Int. Cl. A61K 6/00; A61K 31/015; A61K 31/095

(52) U.S. Cl. CPC A61K 6/0035; A61K 31/095; A61K 6/0044; A61K 31/015

(57) ABSTRACT

A composition and method for use in a dental treatment, such as root canal. The composition eliminates the need for uncomfortable and time-consuming needle injections before application of prep composition. The composition is a mixture of a lubricant, a disinfecting agent, a chelating agent and an anesthetic, preferably articaine, and may also include epinephrine. A method for preparing an area of the patient's mouth for the dental treatment, such as a root canal, is achieved by preparing the disclosed composition and administering it to the area of the patient's mouth to be treated. The composition provides simultaneous antibacterial action and numbing action with a one-step application.

### RELATED APPLICATION

The present application claims the filing benefit under 35 U.S.C. 119(e) of U.S. Provisional Application No. 62/345,952 titled “Anesthetic And RC-Prep Composition” filed on Jun. 6, 2016 (the '952 application). The '952 application is hereby incorporated by reference.

### TECHNICAL FIELD OF THE INVENTION

The present composition relates to anesthetic used for dental procedures. Specifically, the composition relates to a root canal preparation and anesthetic combination.

### BACKGROUND OF THE INVENTION

Root canal therapy is a treatment used to repair and save a tooth that is badly decayed or infected. It is performed when the pulp which is composed of nerves and blood vessels in the tooth becomes infected or damaged. During root canal therapy, the pulp is removed, and the inside of the tooth is cleaned, filled with gutta-percha and sealed.

When the pulp is damaged, it breaks down, and bacteria begin to multiply within the pulp chamber. The bacteria and other dying pulp remnants can cause an infection or abscessed tooth. An abscess is a puss-filled pocket that forms at the end of a tooth's root.

A root canal preparation may typically include a disinfecting agent, a chelating agent, and a lubricant, all in a suitable base. One commercially available root canal preparation lubricant is sold under the trade name RC-PREP®, and available from Premier Dental Products of Plymouth Meeting, Pennsylvania. RC-PREP® is a combination of carbamide peroxide (CP)(urea peroxide) and ethylenediaminetetraacetic acid (EDTA), and has been used for eradication of infectious agents, debridement of deteriorated pulpal tissue and enlargement of an infected root canal wall for preparation of root canal treatment. Although RC-PREP® has been in use since 1969, it has never been used to introduce pain-numbing anesthetic to a treatment area.

Instead, the standard endodontic practice for root canal is to force anesthetic into the canal with a needle until the area becomes numb. Injection of an anesthetic creates an added difficulty for a doctor, and it can be an extremely uncomfortable if not a painful procedure for the patient. Injection directly into the root canal creates pressure inside the tooth chamber forcing debris and bacteria deeper into the canal and into the bone. The pressure of the injection is also a source of great pain and trauma to the patient if the pulp is even partially vital.

Further, infection typically affects the pH of the tooth canal. The altered pH can have a negative effect on the efficacy of anesthetic applied in the conventional manner.

Until the invention of the present application, these problems in the prior art went either unnoticed or unsolved by those skilled in the art. The present invention provides a composition and method of application which, in accordance with one or more aspects of an exemplary embodiment, may perform one or more functions in a root canal treatment without creating pressure inside the pulp chamber and without losing efficacy of the anesthetic.

### SUMMARY OF THE INVENTION

There is disclosed herein an improved composition for use in a root canal procedure which may address one or more shortcomings of prior root canal preparations and/or procedures while affording additional structural and/or operating advantages.

Generally speaking, the disclosed composition comprises a mixture of a dental lubricant, a disinfecting agent such as a peroxide, and a chelating agent, in a suitable base which is preferably a water soluble base, along with a medically effective amount of an anesthetic added to the mixture which will be applied to a hand file or rotary instrument whipped into the canal to apex of the tooth. In embodiments, the disinfecting agent can be an oxidizing agent. In embodiments, the dental chelating agent can also act to stabilize the disinfecting agent in the composition. More specifically, the mixture may comprise cetyl alcohol, urea peroxide, and EDTA in a water soluble base, in combination with the medically effective amount of an anesthetic added to the mixture in the water soluble base.

In specific embodiments, the composition may further comprise epinephrine, which causes vasoconstriction to increase the duration and quality of anesthetic, in a medically effective amount. Without intending to be bound by theory, it is believed that the combination of epinephrine with an anesthetic increases the time the numbing effect of the anesthetic lasts. Further, in specific embodiments the anesthetic, with or without epinephrine, may be selected from the group of products consisting of articaine, lidocaine, mepivacaine, bupivacaine, cinchocaine (i.e., dibucaine), etidocaine, prilocaine, etidocaine, ropivacaine, levobupivacaine, and other -caine products, including but not limited to amino amide-type anesthetics and amino ester-type anesthetics. Preferably, the anesthetic is articaine.

In some preferred forms, the lubricant may be cetyl alcohol, and the disinfecting agent may be urea peroxide (i.e., carbamide peroxide), hydrogen peroxide, or sodium percarbonate, and the chelating agent may be EthyleneDiamineTetracetic Acid (EDTA). Other forms of lubricants, disinfecting agents, and chelating or stabilizing agents may prove suitable.

In still other embodiments, the composition preferably has a viscosity in the range of from about 1000-100,000 cps, at room temperature (20° Celsius). Most preferably, the viscosity is in the range of from about 1000 cps to about 15,000 cps, at room temperature.

There is also disclosed herein a unique method for preparing an area in a patient's mouth for a dental treatment.

Generally speaking, the method comprises the steps of first preparing a composition comprising a mixture of glycol, urea peroxide and EDTA in a water soluble base, and a medically effective amount of an anesthetic added to the mixture in the water soluble base, then applying the composition to an area of a patient's mouth to be treated. Preferably, a sufficient time passes after applying the composition to allow for simultaneous numbing and cleaning of the area by the composition. The method is unique due to the fact that the step of applying the numbing anesthetic is accomplished without the use of a needle injection into the patient's gums. Instead the composition can be applied directly to the area to be treated, for example, by syringe or other applicator system. In a preferred embodiment, the step of applying comprises squirting the composition onto the area to be treated.

These and other aspects of the invention may be understood more readily from the following description and the appended drawings.

### DETAILED DESCRIPTION OF THE INVENTION

While this invention is susceptible of embodiments in different forms, there is described in detail at least one preferred embodiment of the invention with the understanding that the present disclosure is to be considered as an exemplification of the principles of the invention and is not intended to limit the broad aspect of the invention to any of the specific embodiments described.

Generally speaking, a composition for the pre-treatment for a root canal procedure comprises root canal preparation lubricant and a medically effective amount of an anesthetic. Root canal preparation lubricant is a chemo-mechanical preparation used for root canals that includes a formulation of cetyl alcohol, urea peroxide and EDTA in a special water soluble base to help remove calcifications and lubricate canals to permit more efficient instrumentation. It also allows reamers and rotary files to move easily without binding. The uses and purposes of root canal preparation lubricant are well-known to those skilled in the art, so such details will not be further described in this paper. As previously noted, RC Prep® is a preferred lubricant and is manufactured and sold by Premier Dental of Plymouth Meeting, Pa.

Applied in the usual manner, root canal preparation lubricant provides for eradication of infectious agents, debridement of deteriorated pulpal tissue and enlargement of an infected root canal wall. The anesthetic, introduced in the same manner as a component of the root canal preparation lubricant, has the benefit of numbing the canal of the tooth, which is the source of nerve pain, saving the doctor time and the patient unnecessary discomfort. The immediate relief of pain saves the doctor time because the patient gets numb much quicker. As a result of the quicker numbness, the patient is more comfortable and does not receive as many external anesthetic nerve blocks and requires less anesthetic.

The disclosed composition can include a lubricant, a disinfecting agent, a chelating agent, and an anesthetic.

Lubricating agents are well known in the art. Lubricating agents can provide one or more advantages such as serving as a lubricant for instruments during the root canal, serve as humectant and inhibit the composition from drying out, and inhibiting the oxidation of EDTA by a peroxide-based disinfecting agent. Lubricating agents can include, but are not limited to, food-grade propylene glycol, polypropylene glycol, hexylene glycol, butylene glycol, glycerol, and sugar polyols such as sucrose and sorbitol. According to published information, RC-PREP® contains cetyl alcohol 1-10%, EDTA 10-30% and hydrogen peroxide-urea 1-10%.

Disinfecting agents are known in the art. In general, disinfecting agents are antimicrobial agents that are destroy microorganisms and can include alcohols, aldehydes, oxidizing agents, phenolics, quaternary ammonium compounds, and heavy metals. In embodiments, the disinfecting agent can be an oxidizing agent. Oxidizing agents act by oxidizing the cell membrane of microorganisms, which results in a loss of structure and leads to cell lysis and death. Examples of oxidizing disinfecting agents include, but are not limited to, hydrogen peroxide, hydrogen peroxide adducts such as urea peroxide and sodium percarbonate, peracetic acid, dilute sodium hypochlorite, chloramine, chlorine dioxide, and dilute iodine. In embodiments, the oxidizing disinfecting agent is selected from urea peroxide and sodium percarbonate.

The chelating agent of the composition can be any organic substance that forms a stable complex with metal ions. Chelating agents can provide one or more advantages such as demineralizing dental hard tissues of the superficial smear layer, and or stabilizing a hydrogen peroxide adduct in the composition by binding and inhibiting any catalytically active heavy metal ions in the composition. Examples of chelating agents include, but are not limited to EDTA, diethyltriaminepentaacetic acid (DTPAC), ethylene glycol-bis(β-aminoethyl ether)-N,N,N′,N′-tetraacetic acid (egtazic acid, EGTA).

The disclosed composition can also include a water-soluble base as a carrier to modify the viscosity of the composition and promote application of the composition to the treatment area. The water-soluble base can be any water-soluble base that is a viscous liquid, ointment, or gel, to allow placement in the treatment area, that does not interact with the lubricant, disinfecting agent, chelating agent, or anesthetic. Thus, the water-soluble base can be inert to the active ingredients of the composition. In embodiments, the water-soluble base can provide added lubricity to the composition, to help lubricate the instruments during treatment. Suitable water-soluble bases can include, but are not limited to, water-soluble linear polymers of polyethylene glycol (PEGs), methoxypolyethylene glycol (mPEGs), and combinations thereof, for example, water-soluble CARBOWAX™ PEGs and CARBOWAX™ mPEGs available from The Dow Chemical Company (Midland, Mich.).

The anesthetic of the composition preferably comprises a -caine product, such as lidocaine or articaine, although other -caine products may prove suitable. In embodiments, the anesthetic comprises an amino amide-type anesthetic and/or amino ester-type anesthetics. In embodiments, the anesthetic comprises articaine, lidocaine, mepivacaine, bupivacaine, cinchocaine (i.e., dibucaine), etidocaine, prilocaine, etidocaine, ropivacaine, levobupivacaine, or a combination thereof. Still other anesthetics and other components having similar medical purposes may be used in alternate embodiments of the disclosed composition. The amount of anesthetic used in the composition may vary dependent upon the anesthetic used. However, for articaine, for example, it has been found that a composition which applies an amount within the range of from about 0.001 mL to about 1.8 mL is medically effective for most patients. This is accomplished by using a composition having articaine preferably in a range of about 20 to about 80 percent by volume. More preferably, the articaine comprises about 30 to about 50 percent by volume. Most preferred, the articaine used in the composition is in the range of about 35 to 45 percent by volume.

In a specific embodiment, the composition includes epinephrine in addition to the components listed above. An articaine-epinephrine ratio of about 1:200,000 is preferred, though a ratio as low as 1:100,000 may be useful in some circumstances. Preferably, the epinephrine is introduced into the water soluble base of the root canal preparation lubricant. The root canal preparation lubricant and the anesthetic are combined to form a cream or gel having a viscosity preferably in the range of about 1,000-100,000 cps (at room temperature). Most preferably, the viscosity is in the range of about 1,000-15,000 cps (at room temperature). This viscosity allows the composition to be more readily applied to a specific area of a patient's mouth during a dental procedure.

A preferred method for preparing a patient's mouth for a root canal treatment is also an important feature of the present invention. Generally speaking, the method comprises the steps of first preparing a composition as described above, with an example composition comprising a medically effective amount of an anesthetic combined with carbamide peroxide (CP) (urea peroxide) and EDTA in a water soluble base, then administering the novel composition to a rotary instrument or hand file and then inserting the instrument into the tooth canal. The composition can be administered in several ways, including via a pump, a needless syringe, topical applicator, or the like. The method further comprises the step of allowing the anesthetic to sufficiently numb the area before commencing treatment.

The current procedure is to administer the root canal preparation lubricant to the treatment area. If the patient is in pain, the dentist may give an infiltration or nerve block injection. If the patient is still in pain, as a last resort the dentist may choose to inject anesthetic directly to the tooth canal. However, using this technique has significant disadvantages to the present method. The injection of anesthetic can hydraulically force debris abscess and bacteria out the apex of the tooth where it can become trapped in the bone, leading to higher incidents of infection and/or post-operative pain.

Conversely, using the disclosed composition, including the anesthetic in combination with the EDTA of the root canal preparation lubricant and at the noted viscosity, bacteria does not get hydraulically pushed out the apex of the tooth. Rather, any hidden bacteria are eradicated in place by the EDTA while numbing is also initiated by application of the anesthetic. Without intending to be bound by theory it is believed that the EDTA causes disruption of the outer membrane of gram negative bacteria by chelating divalent magnesium and calcium cations found in the outer membrane, with the consequent loss of substantial amounts of lipopolysaccharide, making cells susceptible to the action of many substances such as detergents, proteases, lipases, and lysozymes. This co-administration of the two components is a time saving prep which results in patients suffering less infection and less post-operative pain. These are additional key benefits of the disclosed composition.

Once a required degree of numbing from the anesthetic in the applied composition is achieved, treatment on the patient can commence.

The matter set forth in the foregoing description is offered by way of illustration only and not as a limitation. While particular embodiments have been described, it will be apparent to those skilled in the art that changes and modifications may be made without departing from the broader aspects of applicants' contribution. The actual scope of the protection sought is intended to be defined in the following claims when viewed in their proper perspective based on the prior art.

1. A composition for a dental treatment procedure, the composition comprising:
a mixture of a lubricant, a disinfectant and a chelating agent; and
an anesthetic.

2. The composition of claim 1, further comprising epinephrine.

3. The composition of claim 1, wherein the anesthetic is selected from the group of products consisting of articaine, lidocaine, mepivacaine, bupivacaine, cinchocaine, etidocaine, prilocaine, etidocaine, ropivacaine, levobupivacaine, and combinations thereof.

4. The composition of claim 3, wherein the anesthetic comprises articaine.

5. The composition of claim 1, wherein the composition has a viscosity in the range of from about 1000-100,000 cps, at about 20° C.

6. The composition of claim 5, wherein the viscosity is in the range of from about 1000 cps to about 15,000 cps, at about 20° C.

7. The composition of claim 1, wherein the anesthetic is provided in an amount in the range of from about 0.5 mL to about 6.0 mL.

8. The composition of claim 1, wherein the anesthetic is provided in the range of from about 30 to about 50 percent by volume.

9. The composition of claim 1, wherein the disinfecting agent comprises an oxidizing disinfecting agent comprising hydrogen peroxide, urea peroxide, sodium percarbonate, or a mixture thereof.

10. The composition of claim 1, wherein the lubricant comprises cetyl alcohol, the disinfecting agent comprises urea peroxide, the chelating agent comprises EDTA, and the anesthetic comprises articaine.

11. The composition of claim 1, further comprising a water-soluble base.

12. A method for preparing an area in a patient's mouth for a dental treatment, the method comprising the steps of:
preparing a composition comprising:
a mixture of glycol lubricant, a disinfecting agent and a chelating agent; and
a medically effective amount of an anesthetic;
applying the composition to an area of a patient's mouth to be treated.

13. The method of claim 12, further comprising the step of waiting a sufficient time after applying the composition to allow for simultaneous numbing and cleaning of the area by the composition.

14. The method of claim 12, wherein the step of applying is accomplished without the use of a needle injection into the patient's gums.

15. The method of claim 12, wherein the step of applying comprises squirting the composition on the area.

16. The method of claim 12, wherein the composition further comprises epinephrine added to the mixture.

17. The method of claim 12, wherein the anesthetic is selected from the group consisting of articaine, lidocaine, mepivacaine, bupivacaine, cinchocaine, etidocaine, prilocaine, etidocaine, ropivacaine, levobupivacaine, and combinations thereof.

18. The method of claim 17, wherein the anesthetic comprises articaine.

19. The method of claim 12, wherein the composition has a viscosity in the range of from about 1000-100,000 cps, at about 20° C.

20. The method of claim 19, wherein the viscosity is in the range of from about 1000 cps to about 15,000 cps, at about 20° C.

21. The method of claim 12, wherein the medically effective amount of anesthetic added to the mixture is in the range of from about 0.5 mL to about 6.0 mL.

22. The method of claim 12, wherein the anesthetic is provided in the range of from about 30 to about 50 percent by volume.

22. A composition for a dental treatment procedure, the composition comprising:
a mixture of cetyl alcohol, urea peroxide, EDTA and epinephrine in a water soluble base; and
articaine added to the mixture in the water soluble base in an amount within the range of from about 0.5 mL to about 6.0 mL at a concentration in the range of from about 30 to 50 percent by volume;
wherein the composition has a viscosity in the range of from about 1000-100,000 cps, at about 20° C.
